(19) United States
(12) Patent Application Publication
(10) Pub. No.: US 2010/0285970 A1
(43) Pub. Date: Nov. 11, 2010

(54) METHODS OF SEQUENCING NUCLEIC ACIDS

(76) Inventor: Floyd D. Rose, Bellevue, WA (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 12/751,164

(22) Filed: Mar. 31, 2010

Related U.S. Application Data
(60) Provisional application No. 61/211,498, filed on Mar. 31, 2009.

(51) Int. Cl. C40B 20/00; C12Q 1/68; C40B 40/06

(52) U.S. Cl. 506/2; 435/6; 506/16

(57) ABSTRACT

Disclosed are high-throughput methods for sequencing nucleic acid, which entail identifying the complete set of SNPs in a genome of interest in comparison to a wild type or reference DNA whose sequence is known or substantially known. The methods may also entail use of solid supports containing colonies of amplified nucleic acid fragments e.g., prepared by digesting genomic nucleic acid having substantially known sequence, wherein the sequence of the fragments at each coordinate is known. The supports, per se, and apparati containing them, are also provided.

### CROSS-REFERENCE TO RELATED APPLICATION

This application claims the benefit of the filing date of U.S. Provisional Patent Application No. 61/211,498 filed Mar. 31, 2009, the disclosure of which is hereby incorporated herein by reference.

### FIELD OF THE INVENTION

This invention pertains to high-throughput methodology that directly identifies previously unidentified sequence alterations in DNA, including specific disease-causing DNA sequences in mammals. The methods of the present invention can be used to identify genetic polymorphisms, to determine the molecular basis for genetic diseases, and to provide carrier and prenatal diagnosis for genetic counseling. Moreover, the present invention allows for the relatively fast sequence determination of an entire genome.

### BACKGROUND OF THE INVENTION

Single nucleotide polymorphisms (SNPs) are the most abundant nucleic acid sequence variation found in nature. It has been estimated that in genomic DNA single base-pair variations may be found at approximately 1200-nucleotide intervals suggesting that there may be 2-3×10⁶ SNPs total. However, since individual genomes will have in common the majority of these SNPs (and are therefore not SNPs relative to each other), the actual number of SNPs when comparing the genomes of two individuals is probably far lower. The human nuclear genome is comprised of −3×10⁹ base pairs of DNA. The nucleotide differences when comparing the genome of one individual to that of another individual is thought to be less than 0.06% of the total. In other words, the primary differences between human genomes are these polymorphisms occurring at single nucleotides.

The total number of SNPs that an individual possesses, as well as their positions in the genome, is different for each individual. Because of their abundance and low mutation rate, SNPs are the markers of choice in association studies to identify the genetic risk factors in common diseases (Risch and Merikangas 1996; Kruglyak 1999). As a result of several large initiatives, several million single base-pair variations have been deposited in public and commercial databases. Although many robust genotyping methods have been developed during the past decade, a major challenge still remains from the standpoints of cost and time needed to obtain the genotypes of numerous samples with respect to potentially hundreds of thousands of SNPs.

Despite being only single nucleotide alterations, SNPs are thought to be markers for human diseases. For example, rs4420638 near ApoE has a powerful association with late-onset Alzheimer's disease and rs333 (aka CCR5Delta32) is a well-known SNP associated with HIV. The ability to easily and rapidly detect such alterations in DNA sequences could be central to the diagnosis of genetic diseases and to the identification of clinically significant variants of disease-causing microorganisms. One method for the molecular analysis of genetic variation involves the detection of restriction fragment length polymorphisms (RFLPs) using the Southern blotting technique (Southern, E. M., J. Mol. Biol., 98:503-517, 1975). Since this approach is relatively cumbersome, new methods have been developed, some of which are based on the polymerase chain reaction (PCR). These include: RFLP analysis using PCR (Chehab et al., Nature, 329:293-294, 1987; Rommens et al., Am. J. Hum. Genet. 46:395-396, 1990), the creation of artificial RFLPs using primer-specified restriction-site modification (Haliassos et al., Nucleic Acids Research, 17:3606, 1989), allele-specific amplification (ASA) (Newton C R et al., Nuc. Acids Res., 17:2503-2516, 1989), oligonucleotide ligation assay (OLA) (Landergren U et al., Science 241:1077-1080, 1988), primer extension (Sokolov B P, Nucl. Acids Res., 18:3671, 1989), artificial introduction of restriction sites (AIRS) (Cohen LB et al., Nature 334:119-121, 1988), allele-specific oligonucleotide hybridization (ASO) (Wallace R B et al., Nucl. Acids Res., 9:879-895, 1981) and their variants. Together with robotics, these techniques for direct mutation and analysis have helped in reducing cost and increasing throughput when only a limited number of mutations need to be analyzed for efficient diagnostic analysis.

These methods are, however, limited in their applicability to complex mutational analysis. For example, in cystic fibrosis, a recessive disorder affecting 1 in 2000-2500 live births in the United States, more than 225 presumed disease-causing mutations have been identified. Furthermore, multiple mutations may be present in a single affected individual, and may be spaced within a few base pairs of each other. These phenomena present unique difficulties in designing clinical screening methods that can accommodate large numbers of sample DNAs.

To achieve adequate detection frequencies for rare mutations using the above methods, large numbers of mutations must be screened. To identify previously unknown mutations within a gene, other methodologies have been developed, including: single-strand conformational polymorphisms (SSCP) (Orita M et al., Proc. Natl. Acad. Sci. USA 86:2766-2770, 1989), denaturing gradient gel electrophoresis (DGGE) (Meyers R M et al., Nature 313:495-498, 1985), heteroduplex analysis (HET) (Keen j. et al., Trends Genet. 7:5, 1991), chemical cleavage analysis (CCM) (Cotton R G H et al., Proc. Natl. Acad. Sci., 85:4397-4401, 1988), and complete sequencing of the target sample (Maxam A M et al., Methods Enzymol. 65:499-560, 1980, Sanger F. et al., Proc. Natl. Acad. Sci. USA 74:5463-5467, 1977). All of these procedures however, with the exception of direct sequencing, are merely screening methodologies. That is, they merely indicate that a mutation exists, but do not specify the exact sequence and location of the mutation. Therefore, identification of the mutation ultimately requires complete sequencing of the DNA sample. For this reason, these methods are incompatible with high-throughput and low-cost routine diagnostic methods. Thus, there is a need in the art for a relatively low cost method that allows the efficient analysis of large numbers of DNA samples for the presence of previously unidentified mutations or sequence alterations.

### SUMMARY OF THE INVENTION

The present invention encompasses high-throughput methods for identifying the complete set of SNPs in a genome of interest in comparison to a wild type or reference DNA whose sequence is known or substantially known. In its broadest aspect, the present method is directed to a method of sequencing nucleic acid, comprising: a) preparing single stranded fragments of a first nucleic acid having a substantially known sequence, wherein each of the fragments has a substantially known sequence; b) preparing single stranded fragments of a second nucleic acid having an unknown sequence; c) contacting the single stranded fragments of a) or copies thereof, and the single stranded fragments of b) or copies thereof under conditions that allow formation of heterohybrid nucleic acid, wherein the heterohybrid nucleic acid is perfectly complementary heterohybrid nucleic acid or heterohybrid nucleic acid containing a mismatch; d) distinguishing formation of heterohybrid nucleic acid containing a mismatch from formation of heterohybrid nucleic acid which is perfectly complementary, and e) determining sequences of the mismatches in d), thus allowing elucidation of the sequence of the second nucleic acid.

In various embodiments, the method may be carried out by any one of the following sequences of steps. For example, the method may entail the steps of:

a) attaching (e.g., covalently) one strand of a restriction fragment derived from DNA of unknown sequence to a solid support; and

b) annealing a labeled oligonucleotide to the immobilized restriction fragment, wherein the oligonucleotide contains a SNP and the specific annealing indicates the presence of a SNP in the restriction fragment.

Alternatively, the method may entail the steps of:

a) attaching (e.g., covalently) and amplifying a restriction fragment derived from DNA of known sequence (reference DNA) to a solid support;

b) annealing the corresponding amplified complementary restriction fragment from a DNA of unknown sequence (target DNA) to the restriction fragment DNA attached to the solid support creating a heterohybrid double strand DNA;

c) cleaving one DNA strand in the either the known or unknown strand of heterohybrid DNA to form a nick in the phosphodiester bond of one of the strands at the site of a mismatch;

d) determining the nucleotide sequence in the vicinity of the nick; and

e) comparing the nucleotide sequence determined in d) with the predetermined known sequence to identify the mismatch and its location.

In a further embodiment, the method may entail the steps of:

a) attaching (e.g., covalently) and amplifying a restriction fragment derived from DNA of unknown sequence (target DNA) to a solid support;

b) annealing the corresponding amplified complementary restriction fragment from a DNA of known sequence (reference DNA) to the restriction fragment DNA attached to the solid support creating a heterohybrid double strand DNA

c) cleaving one DNA strand in the either the known or unknown strand of heterohybrid DNA to form a nick in the phosphodiester bond of one of the strands at the site of a mismatch;

d) determining the nucleotide sequence in the vicinity of the nick; and

e) comparing the nucleotide sequence determined in d) with the predetermined known sequence to identify the mismatch and its location.

In yet a further embodiment, the method may entail the steps of:

a) creating heterohybrid DNA comprised of reference and target DNA in solution;

b) identifying and purifying heterohybrids containing mismatches using mismatch recognition enzymes; and

c) sequencing one or more of the purified mismatch containing heterohybrids.

In practicing the present invention, the target DNA is hybridized under stringent conditions with a reference DNA sample. The hybrids that form may contain mismatch regions, which are recognized and endonucleolytically cleaved on one or both sides of the mismatch region by mismatch recognition protein-based systems. When a single endonucleolytic cleavage occurs on only one side of the mismatch region, one or more exonucleases can be used to form a single-stranded nick or gap. When endonucleolytic cleavage occurs on both the 3′ and 5′ sides of the mismatch region, the single-stranded fragment is released by the action of a helicase to form the single-stranded gap. Determination of the sequence across the gap is achieved in a single step by an enzymatic DNA sequencing reaction using dideoxynucleotides or nucleotides with removable 3′ terminators and DNA polymerase I, DNA polymerase III, T4 DNA polymerase, or T7 DNA polymerase.

In an embodiment of the method, the nick at the site of the mismatch in the heterohybrid DNA is created by an ATE enzyme (“all-type nicking enzyme”) and results in the covalent attachment of the enzyme to the nicked strand. For example, DNA topoisomerase I is a ubiquitous enzyme that relieves DNA torsional stress by introducing a break in the phosphodiester bond between the mismatch nucleotide and the nucleotide immediately on the 5′ side of the mismatch. The enzyme becomes covalently attached to the free 3′ hydroxyl via a phosphotyrosine moiety. Using a fluorescent ATE, the resulting fluorescent signal indicates the presence of a mismatch in a heterohybrid DNA. A greater intensity of the fluorescent signal also indicates that there is more than one mismatch in the DNA fragment. Since ATEs can be strand selective based on local nucleotides, the strand containing the nick can be ultimately identified by comparison to the known sequence (which may be contained in a database), once the sequence in the vicinity of the mismatch is obtained. Covalently bound ATE can be removed by proteolysis or by the activity of a tyrosyl phosphodiesterase. The resulting 3′-phosphorylated nick is reconstituted to a 3′ hydroxyl by polynucleotide kinase phosphatase to be a substrate for DNA polymerase and hence for sequencing.

In another embodiment of the method, the heterohybrid DNA is created from fragments of target and reference DNA that are obtained either directly from fragmented genomic DNA or indirectly from amplified or cloned fragments of genomic DNA. These heterohybrids in solution are then reacted with one or more mismatch repair enzymes under conditions in which the repair enzyme(s) remains attached to the mismatch region of the heterohybrid for a sufficient period of time to allow for further manipulation of the enzyme-DNA complex. Examples of further manipulation include, for example, purification, precipitation, hybridization, and denaturation. In an embodiment, the mismatch repair enzyme is a topoisomerase and the method of attachment to the mismatch region is by covalent attachment of the enzyme to the DNA. In another preferred embodiment, the mismatch repair enzyme is a biotinylated topoisomerase and the further manipulation involves contacting the enzyme-DNA complex with streptavidin attached to a solid support. By this method, heterohybrids containing mismatches can be first identified in solution by enzyme binding and subsequently purified by affinity chromatography. These mismatch-containing heterohybrids can then be used as the starting material for amplification, immobilization, and sequencing.

Typically, the first immobilized DNA sample comprises genomic DNA from a known or substantially known sequence of DNA referred to as the reference standard or reference DNA. The second DNA sample is genomic DNA of unknown sequence, or target DNA, that is processed in the same fashion as the first sample. For example, if the reference DNA was digested with a particular restriction enzyme or enzymes, the target DNA is preferably digested with the same enzyme(s). Alternatively, the first immobilized DNA sample comprises genomic DNA from an unknown sequence of DNA referred to as the target DNA. The second DNA sample is genomic DNA of known or substantially known sequence, or reference DNA, that is processed in the same fashion as the first sample.

The various genetic alterations identified by these methods include additions, deletions, or substitutions of one or more nucleotides. Mismatch recognition, cleavage, and excision systems useful in practicing the invention include without limitation nicking proteins, mismatch repair proteins, nucleotide excision repair proteins, chemical modification of mismatched bases followed by excision repair proteins, and combinations thereof, with or without supplementation with exonucleases as required.

The present invention finds application in the manufacturing of a chip having affixed thereto, directly or indirectly, a plurality (typically in the order of thousands to millions) of restriction fragments or other types of DNA fragments of known sequence, which constitutes another aspect of the present invention. These fragments, which have a known sequence and which may be arranged in known locations on the chip, serve as the annealing templates for similarly processed target or reference DNA. The heterohybrid DNA thus contained on the chip can be used to identify regions where the sequences of the two strands differ and mismatched bases are present. The existence of one or more mismatched bases is determined by enzymatic activity on one or both of the DNA strands at the site of the mismatch. Subsequent sequencing methods at the site of the mismatches will result in the identification of all sequence differences that exist when comparing an unknown DNA to the known sequence of a reference standard.

### DETAILED DESCRIPTION OF THE INVENTION

All patent applications, patents, and literature references cited in this specification are hereby incorporated by reference in their entirety. In case of conflict, the present description, including definitions, will control.

The present invention encompasses high-throughput methods for identifying the DNA sequence of a genome. As used herein, the term high-throughput refers to a system for rapidly assaying large numbers of DNA samples at the same time.

In practicing the methods of the present invention, the unknown genomic DNA sequence is hybridized with genomic DNA of known sequence. A “known DNA sequence” as referred to herein refers to a sequence of nucleotides comprising a gene, a set of genes, or a genome where the nucleotide sequence is substantially or entirely known such that oligonucleotides complementary to repeating units of the gene, set of genes, or genome can be synthesized. Examples of such repeating units include but are not limited to, for example, SNPs and restriction sites. An “unknown DNA sequence” is a gene, set of genes, or a genome that has not yet been sequenced.

The methods of the present invention take advantage of the physico-chemical properties of DNA hybrids between almost-identical (but not completely identical) DNA strands (i.e., heteroduplexes). When a sequence alteration is present, the heteroduplexes contain a mismatch region that is embedded in an otherwise perfectly matched hybrid. According to the present invention, mismatch regions are formed under controlled conditions and are chemically and/or enzymatically modified. The sequences adjacent to, and including, the mismatch are then determined. Depending upon the mismatch recognition method used, the mismatch region may include any number of bases, typically from 1 to about 1000 bases.

The methods of the present invention encompass the steps of:

1) preparing heteroduplexes between a DNA of known sequence (a reference DNA) and a DNA of unknown sequence (a target DNA);

2) cleaving one or both of the DNA strands at mismatches to form a single-stranded nick or gap at the site of the thereby creating a substrate for DNA polymerases and;

3) determining the precise sequence at the site of the mismatch.

These steps are described in detail below.

### PREPARATION OF NUCLEIC ACID COORDINATES ON SOLID SUPPORTS

In accordance with the present invention, the target DNA represents a sample of DNA isolated from an animal or human patient. This DNA may be obtained from any cell source or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, and tissue exudates at the site of infection or inflammation. DNA is extracted from the cell source or body fluid using any of the numerous methods that are standard in the art. It will be understood that the particular method used to extract DNA will depend on the nature of the source. The amount of DNA to be extracted for analysis of human genomic DNA is typically in the range of at least 5 pg (corresponding to about 1 cell equivalent of a genome size of 3×10⁹ base pairs). In some applications, such as, for example, detection of sequence alterations in the genome of a microorganism, variable amounts of DNA may be extracted.

Likewise, the reference DNA may be obtained from any cell source or body fluid. In one embodiment, the reference DNA is obtained from a single cell source for comparison to different target DNAs. A single source of reference DNA could be obtained, for example, from human cells in culture. Alternatively, reference DNA can be obtained from an individual with a particular disease, such as cancer. Reference DNAs obtained from individuals with diagnosed diseases or clinical symptoms or genetic traits could be used to ultimately identify unique SNP profiles associated with diseases, symptoms, or traits. In this way, multiple reference DNAs can be obtained which will each contain the SNP profile relating to a disease, a particular symptom or a trait.

Once extracted, the DNA may be employed without further manipulation. The DNA may be cleaved by one or more restriction enzymes to create discrete restriction fragments. These fragments may be amplified by PCR either before or after attachment to a solid support. The amplified regions may be specified by the choice of particular flanking sequences for use as primers or alternatively the primers can be ligated to the ends of each restriction fragment. Amplification provides the advantage of increasing the amount of either specific DNA or total sequences within the DNA sequence population. The length of DNA sequence that can be amplified typically ranges from 80 by up to about 30 kbp (Saiki et al., 1988, Science, 239:487). Furthermore, the use of amplification primers that are modified by, e.g., biotinylation, can allow for the selective incorporation of the modification into the amplified target DNA.

Nucleic acids which may be amplified according to the methods of the invention include DNA, for example, genomic DNA, cDNA, recombinant DNA or any form of synthetic or modified DNA, RNA, mRNA or any form of synthetic or modified RNA. The nucleic acids may vary in length and may be fragments or smaller parts of larger nucleic acid molecules. The nucleic acid to be amplified is typically at least 50 base pairs in length and in some embodiments about 30,000 base pairs in length. The nucleic acid to be amplified may have a known or unknown sequence and may be in a single or double-stranded form. The nucleic acid to be amplified may be derived from any source.

“Nucleic acid template” as used herein refers to an entity that includes or contains the nucleic acid to be amplified or sequenced. As outlined below the nucleic acid to be amplified or sequenced can also be provided in a double stranded form. Thus, “nucleic acid templates” of the invention may be single or double stranded nucleic acids. The nucleic acid templates to be used in the method of the present invention can be of variable lengths, typically at least 50 base pairs in length and in some embodiments about 30,000 base pairs in length. The nucleotides making up the nucleic acid templates may be naturally occurring or non-naturally occurring nucleotides. The nucleic acid templates of the invention not only comprise the nucleic acid to be amplified but may in addition contain at the 5′ and 3′ end short sequences that are complementary to synthetic oligonucleotides

In one embodiment, either the reference DNA or target DNA, with or without prior amplification, is bound to a solid-phase matrix. This allows the simultaneous processing and screening of a large number of restriction fragments. Non-limiting examples of matrices suitable for use in the present invention include nitrocellulose or nylon filters, glass beads, magnetic beads coated with agents for affinity capture, treated or untreated microtiter plates, and the like. It will be understood by a skilled practitioner that the method by which the DNA is bound to the matrix will depend on the particular matrix used. For example, binding to nitrocellulose can be achieved by simple adsorption of DNA to the filter, followed by baking the filter at 75°-80° C. under vacuum for 15 min-2 h. Alternatively, charged nylon membranes can be used that do not require any further treatment of the bound DNA. Beads and microtiter plates that are coated with avidin can be used to bind target DNA that has had biotin attached (via, e.g., the use of biotin-conjugated PCR primers.) In addition, antibodies can be used to attach DNA to any of the above solid supports by coating the surfaces with the antibodies and incorporating an antibody-specific hapten into the target DNA.

In one embodiment, methods for attachment to a solid support followed by amplification and sequencing of at least one nucleic acid includes the following steps as described in U.S. Pat. No. 7,115,400: (1) forming at least one nucleic acid template comprising the nucleic acid(s) to be amplified or sequenced, wherein said nucleic acid(s) to be amplified or sequenced, wherein said nucleic acid(s) contains at the 5′ end an oligonucleotide sequence Y and at the 3′ end an oligonucleotide sequence Z and, in addition, the nucleic acid(s) carry at the 5′ end a means for attaching the nucleic acid(s) to a solid support; (2) mixing said nucleic acid template(s) with one or more colony primers X, which can hybridize to the oligonucleotide sequence Z and carries at the 5′ end a means for attaching the colony primers to a solid support, in the presence of a solid support so that the 5′ ends of both the nucleic acid template and the colony primers bind to the solid support; and (3) performing one or more nucleic acid amplification reactions on the bound template(s), so that nucleic acid colonies are generated and optionally, performing at least one step of sequence determination of one or more of the nucleic acid colonies generated. When this technique is used to randomly create discrete populations of amplified nucleic acid on a solid support, the amplified nucleic acids are referred to as colonies.

Once each restriction fragment is amplified on the solid support in this fashion, sufficient sequencing is performed using preferably the removable 3′ fluorescent terminator technique, or any of the other sequencing techniques to allow for the identification of the particular fragment comprising each colony.

In some embodiments, small fragments of synthetic DNA (20-100 bp) that are complementary to sequences in the reference or target DNA adjacent to restriction sites are attached by their 5′ ends using any of the methods heretofore described. In some embodiments, these synthetic fragments include the entire set of sequences in the reference or target genome that are complementary to the 3′ region of all restriction sites for cleaving by one or more restriction enzymes and are referred to as “A” primers. The oligonucleotide sequence complementary to one end of the restriction fragment is therefore referred to herein as the “A” primer. The “A” primer attached by its 5′ end to a solid support as used herein refers to an entity which contains an oligonucleotide sequence which is capable of hybridizing to a complementary sequence and initiating a specific polymerase reaction using an annealed template DNA strand. The sequence containing the coordinate primer is chosen such that it has maximal hybridizing activity with its complementary sequence and very low non-specific hybridizing activity to any other sequence.

The oligonucleotide sequences containing the “A” primers are of known sequence and can be of variable length and are attached to a solid support by their 5′ end and are complementary to the region of DNA in a genome that is on the 3′ side of a specific restriction site and may include the entire restriction site. Oligonucleotide sequence “A” for use in the methods of the present invention is typically at least five nucleotides in length, and in some embodiments between 5 and about 100 nucleotides in length, or in some other embodiments approximately (or “about”) 20 nucleotides in length. Naturally occurring or non-naturally occurring nucleotides may be present in the oligonucleotide sequence “A”.

In a further embodiment, synthetic DNAs are each attached to the solid support to create a grid whereby the coordinates of any particular synthetic sequence on the grid are known. In one embodiment, the restriction site is Sbf1and the average distance between restriction sites is about 30 kbp. In this instance, the total number of synthetic fragments that are complementary to the 3′ sequence adjacent to the Sbf1 restriction site would be approximately 100,000. Therefore, in this embodiment, the “A” primers contain the Sbf1 site and approximately 14 additional nucleotides complementary to the 3′ sequence adjacent to the Sbf1 site. One of skill in the art would understand that a restriction fragment results from the cleavage at two restriction sites both of which contain regions that are 3′ to the site. In preparing the “A” primers, only one strand sequence is used to synthesize complementary “A” primer oligonucleotides. The equivalent sequence located at the opposite end of the restriction fragment is complementary to what is referred to as the “B” primer and is used to initiate DNA amplification or DNA extension. The placement of “A” primers on the solid support to create a grid is done in a non-random fashion such that the positional coordinate of each of the “A” primer sequences is known.

The non-covalent annealing of complementary restriction fragments to the approximately 100,000 synthetic fragments involves separating the two strands of the restricted reference DNA using, for example, heat. One of each of the strands derived from the restriction fragments is then annealed at a lower temperature to each of the immobilized synthetic DNAs. Typically, the immobilized synthetic DNA sequence is present in a 10-fold to 10,000-fold molar excess compared to the complementary fragment DNA derived from the restricted reference DNA. The immobilized synthetic DNA, when annealed to the restriction fragment strand, creates a substrate for a polymerase that can extend the synthetic strand using the annealed restriction fragment as a template. The “A” primer extended synthetic strand is therefore a covalently attached and immobilized complementary copy of the restriction fragment strand that had been annealed to the original immobilized “A” primer. The original DNA restriction fragment strand is washed away after heating. The resulting chip grid thereby contains an entire genome of DNA restriction fragments that are positioned at the above-mentioned discrete coordinates by virtue of annealing and extension of the approximately 100,000 immobilized synthetic DNAs previously described. These DNAs are now ready for amplification to increase the amount of DNA at each coordinate location. These extended DNA fragments are collectively called the “C” strands, referring to their complementarity to one strand of the original DNA restriction fragment.

Amplification of the “C” strand is initiated by annealing to it a synthetic oligonucleotide containing both the “B” primer as well as the “A” primer. The resulting synthetic primer “AB” is in the order 5′ “AB”3′. Oligonucleotide sequence “AB” is of a known sequence and can be of variable length. Oligonucleotide sequence “AB” for use in the methods of the present invention is typically at least five nucleotides in length, and in some embodiments between 5 and about 100 nucleotides in length or in yet other embodiments, approximately 40 nucleotides in length. Naturally occurring or non-naturally occurring nucleotides may be present in the oligonucleotide sequence “AB”. Oligonucleotide sequence “AB” is designed so that it also hybridizes with a section of the template DNA that is adjacent to a restriction fragment. The oligonucleotide sequences “A” and “AB” are typically contained at the 5′ and 3′ ends respectively, of a nucleic acid restriction fragment template but need not be located at the extreme ends of the template. For example, although the oligonucleotide sequences “A” and “AB” are typically located at or near the 5′ and 3′ ends (or termini) respectively of the nucleic acid templates (for example within 0 to about 100 nucleotides of the 5′ and 3′ termini) they may be located further away (e.g., greater than about 100 nucleotides) from the 5′ or 3′ termini of the nucleic acid template. The oligonucleotide sequences “A” and “AB” may therefore be located at any position within the nucleic acid template providing the sequences “A” and “AB” are on either side, i.e., flank, the nucleic acid sequence which is to be amplified.

“Nucleic acid template” as used herein also includes an entity which comprises the nucleic acid to be amplified or sequenced in a double-stranded or single-stranded form. When the nucleic acid template is “C”, the sequence “A” and the sequence complementary to “B” are contained at the 5′ and 3′ ends respectively, of the “C” strand.

Amplification of the “C” strand is accomplished using an “amplification initiator” synthetic DNA primer composed of the two sequences “A” and “B” described above. The 5′ region of the amplification initiator primer is the same as the “A” primer sequence whereas the 3′ region of the amplification initiator primer contains the sequence complementary to the “B” primer at the 3′ end of the “C” strand. This 3′ end of the “C” strand is derived from the reference DNA database and is 3′ proximal to the restriction site used to create the original reference DNA restriction fragments. As with the “A” sequence, if the restriction enzyme recognizes a sequence every approximately 30,000 nucleotides, the number of “B” primer complementary sequences in a genome would be approximately 100,000. Therefore the number of amplification initiator primers would also be −100,000. The amplification initiator primer is referred to as the “AB” primer and contains of from 20 to 100 nucleotides. In an embodiment, the “AB” primer contains a dideoxynucleotide at its 3′ end and is thereby not a substrate for a polymerase catalyzed extension. The “B” portion of the “AB” primer anneals to its complementary sequence on the “C” strand and provides a template for further extension of the “C” strand. In the presence of a DNA polymerase, extension of the “C” strand using the “AB” primer as a template results in the introduction of a sequence complementary to the “A” primer. After the “C” strand extension, the “AB” primer is removed by heating and washing the solid support. Amplification of the “C” strand proceeds without the introduction of any additional primers by virtue of the continual annealing and extension of the “A” primer using the “C” strand as template or the complementary copy of the “C” strand as template. Each newly synthesized strand (“C” or the complementary copy of “C”) is covalently attached to the solid support by virtue of a phosphodiester bond to the “A” primer.

In a further embodiment of the invention, the amplified DNA containing “C” or the complement of “C” in a colony or coordinate is subjected to one additional round of polymerase primer extension. This primer extension uses for example either the “C” strand or the “C” strand complement as a template. Priming a polymerase reaction on these templates therefore uses either the “A” primer or the “AB” primer or oligonucleotides containing only the “B” region of the “AB” primers. In this instance the “B” primer has a 3′ hydroxyl group. The resulting population of non-covalent, single stranded nucleic acids derived from the “B” primer extension can then be melted from the template strands and transferred a new solid support while maintaining the discrete positional identification of each of the restriction fragments. A variety of methods can be employed to accomplish this type of “replica transfer”. For example, if the primer used in the final polymerase reaction is biotinylated and the new solid support is coated with avidin or streptavidin, contacting the two solid support surfaces at a temperature sufficient to separate DNA strands, followed by a reduction in temperature, will release the newly synthesized strands from the first solid support and transfer them to the new solid support. The resulting new solid support is a single stranded replica of the original solid support containing only the strand complementary to “C” resulting from the extension of “B” primers. Other primers, containing sequences complementary to “C” or its complementary strand, or to a subset of the “C” strands or complement, can be used to selectively amplify all or some of the clusters or portions of the clusters. When annealing target DNA to any of these single stranded arrays, only one complementary strand in the target DNA will bind to the specific colony or coordinate replica.

“Solid support” as used herein refers to any solid surface to which nucleic acids can be covalently attached, such as for example latex beads, dextran beads, polystyrene, polypropylene surface, polyacrylamide gel, gold surfaces, glass surfaces and silicon wafers. Preferably the solid support is a glass surface.

“Means for attaching nucleic acids to a solid support” as used herein refers to any chemical or non-chemical attachment method including chemically-modifiable functional groups. “Attachment” relates to immobilization of nucleic acid on solid supports by either a covalent attachment or via irreversible passive adsorption or via affinity between molecules (for example, immobilization on an avidin-coated surface by biotinylated molecules). The attachment must be of sufficient strength that it cannot be removed by washing with water or aqueous buffer under DNA-denaturing conditions.

“Chemically-modifiable functional group” as used herein refers to a group such as for example, a phosphate group, a carboxylic or aldehyde moiety, a thiol, or an amino group.

“Nucleic acid coordinate” or “coordinate” as used herein refers to a discrete area containing multiple copies of a nucleic acid strand or a synthetic oligonucleotide of known sequence e.g., sequences comprising “A” primers. Multiple copies of the complementary strand to the nucleic acid strand may also be present in the same coordinate. The multiple copies of the nucleic acid strands making up the coordinates are generally immobilized on a solid support and may be in a single or double stranded form. The nucleic acid colonies of the invention can be generated in different sizes and densities depending on the conditions used. Nucleic acid coordinates are distinguished from nucleic acid colonies by virtue of having the positions of specific oligonucleotides or nucleic acids on the solid support predetermined by the mechanical placement pf “A” primers at defined locations. Nucleic acid colonies, as described above, are a random array of amplified nucleic acids amplified from a lawn of colony primers. For convenience, both nucleic acid coordinates and colonies are referred to as clusters.

The size of cluster is typically about 0.2 μm to about 6 μm, and in some embodiments about 0.3 μm to about 4 μm. The density of nucleic acid cluster for use in the methods of the invention typically ranges from about 10,000/mm² to about 100,000/mm². It is believed that higher densities, for example, about 100,000/mm² to about 1,000,000/mm², and about 1,000,000/mm² to about 10,000,000/mm² may be achieved.

Preferably the attachment of the oligonucleotide primer as well as the extended nucleic acid template on the solid support is thermostable at the temperature to which the support may be subjected to during the nucleic acid amplification reaction, for example temperatures of up to approximately 100° C., for example approximately 94° C. Preferably the attachment is covalent in nature.

In a yet further embodiment of the invention, the covalent binding of the synthetic primers to the solid support is induced by a crosslinking or grafting agent such as for example 1-ethyl-3-(3-dimethylaminopropyl)-carbodiimide hydrochloride (EDC), succinic anhydride, phenyldiisothiocyanate or maleic anhydride, or a hetero-bifunctional crosslinker such as for example m-maleimidobenzoyl-N-hydroxysuccinimide ester (MBS), N-succinimidyl[4-iodoacethyl]aminobenzoate (SIAB), Succinimidyl 4-[N-maleimidomethyl]cyclohexane-1-carboxylate (SMCC), N-y-maleimidobutyryloxy-succinimideester (GMBS), Succinimidyl-4-[p-maleimidophenyl]butyrate (SMPB) and the sulfo (water-soluble) corresponding compounds. Preferred crosslinking reagents for use in the present invention are s-SIAB, s-MBS and EDC. s-MBS is a maleimide-succinimide hetero-bifunctional cross-linker and s-SIAB is an iodoacethyl-succinimide hetero-bifunctional cross-linker. Both are capable of forming a covalent bond respectively with SE groups and primary amino groups. EDC is a carbodiimide-reagent that mediates covalent attachment of phosphate and amino groups.

In a yet further embodiment of the invention the solid support has a derivatized surface. In a yet further embodiment the derivatized surface of the solid support is subsequently modified with bifunctional crosslinking groups to provide a functionalized surface, preferably with reactive crosslinking groups.

“Derivatized surface” as used herein refers to a surface which has been modified with chemically reactive groups, for example amino, thiol or acrylate groups.

“Functionalized surface” as used herein refers to a derivatized surface which has been modified with specific functional groups, for example the maleic or succinic functional moieties.

In the method of the present invention, to be useful for certain applications, the attachment of primers to a solid support has to fulfill several requirements. The ideal attachment should not be affected by either the exposure to high temperatures and the repeated heating/cooling cycles employed during the nucleic acid amplification procedure. Moreover the support should allow the attached colony primers to achieve a density of at least 1 fmol/mm², preferably at least 10 fmol/mm², more preferably between about 30 to about 60 fmol/mm². The ideal support should have a uniformly flat surface with low fluorescence background and should also be thermally stable (non-deformable). Solid supports, which allow the passive adsorption of DNA, as in certain types of plastic and synthetic nitrocellulose membranes, are less preferred. Finally, the solid support should be disposable (and thus relatively inexpensive as well).

For these reasons, although the solid support may be any solid surface to which nucleic acids can be attached, such as for example latex beads, dextran beads, polystyrene, polypropylene surface, polyacrylamide gel, gold surfaces, glass surfaces and silicon wafers, preferably the solid support is a glass surface and the attachment of nucleic acids thereto is a covalent attachment.

The covalent binding of the oligonucleotide primers to the solid support can be carried out using standard techniques. For example, epoxysilane-amino covalent linkage of oligonucleotides on solid supports such as porous glass beads has been widely used for solid phase in situ synthesis of oligonucleotides (via a 3′ end attachment) and has also been adapted for 5′ end oligonucleotide attachment. Oligonucleotides modified at the 5′ end with carboxylic or aldehyde moieties have been covalently attached on hydrazine-derivatized latex beads (Kremsky et al 1987).

Other approaches for the attachment of oligonucleotides to solid surfaces use crosslinkers, such as succinic anhydride, phenyldiisothiocyanate (Guo et al 1994), or maleic anhydride (Yang et al 1998). Another widely used crosslinker is 1-ethyl-3-(3-dimethylamonipropyl)-carbodiimide hydrochloride (EDC). EDC chemistry was first described by Gilham et al (1968) who attached DNA templates to paper (cellulose) via the 5′ end terminal phosphate group. Using EDC chemistry, other supports have been used such as, latex beads (Wolf et al 1987, Lund et al 1988), polystyrene microwells (Rasmussen et al 1991), controlled-pore glass (Ghosh et al 1987) and dextran molecules (Gingeras et al 1987). The condensation of 5′ amino-modified oligonucleotides with carbodiimide mediated reagent have been described by Chu et al (1983), and by Egan et al (1982) for 5′ terminal phosphate modification group.

The yield of oligonucleotide attachment via the 5′ termini using carbodiimides can reach 60%, but non-specific attachment via the internal nucleotides of the oligonucleotide is a major drawback. Rasmussen et al (1991) have enhanced to 85% the specific attachment via the 5′ end by derivatizing the surface using secondary amino groups.

More recent publications report the advantages of the hetero-bifunctional cross-linkers. Hetero- or mono-bifunctional cross-linkers have been widely used to prepare peptide carrier conjugate molecules (peptide-protein) in order to enhance immunogenicity in animals (Peeters et al 1989). Most of these grafting reagents have been described to form stable covalent links in aqueous solution. These crosslinking reagents have been used to bind DNA onto a solid surface at only one point of the molecule.

Chrisey et al (1996) have studied the efficiency and stability of DNA solid phase attachment using 6 different hetero-bifunctional cross-linkers wherein the attachment occurs only at the 5′ end of DNA oligomers modified by a thiol group. This type of attachment has also been described by O'Donnell-Maloney et al (1996) for the attachment of DNA targets in a MALDI-TOF sequence analysis and by Hamamatsu Photonics F.K. company (EP-A-665293) for determining base sequence of nucleic acid on a solid surface.

There are very few reports of studies concerning the thermal stability of the attachment of the oligonucleotides to the solid support. Chrisey et al (1996) reported that with the Succinimidyl-4-[p-maleimidophenyl]butyrate (SMPB) cross-linker, almost 60% of molecules are released from the glass surface during heat treatment. But the thermal stability of the other reagents has not been described.

In order to generate nucleic acid clusters via the solid phase amplification reaction as described in the present application, oligonucleotide primers need to be specifically attached at their 5′ ends to the solid surface, preferably glass. Briefly, the glass surface can be derivatized with reactive amino groups by silanization using amino-alkoxy silanes. Suitable silane reagents include aminopropyltrimethoxysilane, aminopropyltriethoxysilane and 4-aminobutyltriethoxysilane. Glass surfaces can also be derivatized with other reactive groups, such as acrylate or epoxy using epoxysilane, acrylatesilane and acrylamidesilane. Following the derivatization step, nucleic acid molecules or oligonucleotides having a chemically modifiable functional group at their 5′ end, for example phosphate, thiol or amino groups are covalently attached to the derivatized surface by a crosslinking reagent such as those described above.

Alternatively, the derivatization step can be followed by attaching a bifunctional cross-linking reagent to the surface amino groups thereby providing a modified functionalized surface. Nucleic acid molecules (colony primers or nucleic acid templates) having 5′-phosphate, thiol or amino groups are then reacted with the functionalized surface forming a covalent linkage between the nucleic acid and the glass.

Potential cross-linking and grafting reagents that can be used for covalent DNA/oligonucleotide grafting on the solid support are described above.

The oligonucleotide primers are generally modified at the 5′ end by a phosphate group or by a primary amino group (for EDC grafting reagent) or a thiol group (for s-SIAB or s-MBS linkers).

Thus, another aspect of the invention provides a solid support, to which there is attached a plurality of oligonucleotide primers or nucleic acids as described above. Preferably a plurality of nucleic acid templates are attached to the solid support, such as glass. Preferably the attachment of the oligonucleotide primers to the solid support is covalent. By performing one or more rounds of nucleic acid amplification on the annealed or immobilized nucleic acid template(s) using methods as described above, nucleic acid clusters of the invention may be formed. Thus, in some embodiments, the support contains one or more nucleic acid cluster of the invention.

A yet further aspect of the invention provides the use of a derivatized or functionalized support, prepared as described above, in methods of nucleic acid amplification or sequencing. Such methods of nucleic acid amplification or sequencing include the methods of the present invention.

A yet further aspect of the invention provides an apparatus for carrying out the methods of the invention or an apparatus for producing a solid support containing nucleic acid clusters of the invention. Such apparatus might include for example a plurality of nucleic acid templates and oligonucleotide primers of the invention bound, preferably covalently, to a solid support as outlined above, together with a nucleic acid polymerase, a plurality of nucleotide precursors such as those described above, a proportion of which may be labeled, and a means for controlling temperature. Alternatively, the apparatus might include for example a support comprising one or more nucleic acid colonies of the invention. Preferably the apparatus also contains a detecting means for detecting and distinguishing signals from individual nucleic acid clusters arrayed on the solid support according to the methods of the present invention. For example such a detecting means might contain a charge-coupled device operatively connected to a magnifying device such as a microscope as described above.

Preferably any apparati of the invention are provided in an automated form.

The present application is believed to provide a solution to current and emerging needs that face the biotechnology industry and particularly the fields of genomics, pharmacogenomics, drug discovery, food characterization and genotyping. Thus the method of the present invention has potential application in for example: nucleic acid sequencing and re-sequencing, diagnostics and screening, gene expression monitoring, genetic diversity profiling, whole genome polymorphism discovery and scoring, the creation of genome slides (whole genome of a patient on a microscope slide) and whole genome sequencing.

Thus the present invention may be used to carry out nucleic acid sequencing and re-sequencing, where for example a selected number of genes are specifically amplified into clusters for complete DNA sequencing. Gene re-sequencing allows the identification of all known or novel genetic polymorphisms of the investigated genes. Industrial applications include medical diagnosis and genetic identification of living organisms.

The methods of the invention can be used to generate nucleic acid clusters. Thus, a further aspect of the invention provides one or more nucleic acid clusters. A nucleic acid cluster of the invention may be generated from a single immobilized oligonucleotide or nucleic acid template of the invention. The method of the invention allows the simultaneous production of a number of such nucleic acid clusters, each of which will contain different immobilized oligonucleotides and wherein at each cluster the particular oligonucleotide sequence is known.

Thus, a yet further aspect of the invention provides a plurality of nucleic acid templates containing the nucleic acids to be amplified, wherein the nucleic acids contain at their 5′ ends an oligonucleotide sequence complementary to the “A” primer and at the 3′ end an oligonucleotide sequence complementary to the “B” region of the “AB” primer. Preferably the nucleic acid templates are hybridized to a plurality of synthetic primers “A” which carry at the 5′ end a means for attaching the oligonucleotides to a solid support. Preferably the plurality of nucleic acid templates is covalently bound to a solid support due to the attachment of oligonucleotide primers to the solid support.

The nucleic acids to be amplified can be obtained using methods well known and documented in the art. For example, by obtaining a nucleic acid sample such as, total DNA, genomic DNA, cDNA, total RNA, mRNA etc. by methods well known and documented in the art and generating fragments therefrom by, for example, limited restriction enzyme digestion or by mechanical means.

Typically, the nucleic acid to be amplified is first obtained in double stranded form. If at least part of the sequence of the nucleic acid to be amplified is known, the nucleic acid template containing oligonucleotide sequences complementary to “A” and “B” at the opposite ends of the DNA, may be generated by PCR using appropriate PCR primers which include sequences specific to the nucleic acid to be amplified. In one embodiment, the nucleic acid amplification is done using “A” and “B” primers prior to annealing and attachment of the nucleic acid to a solid support.

Before annealing to oligonucleotide attached to the solid support, it can be made into a single stranded form using methods which are well known and documented in the art, for example by heating to approximately 94° C. and quickly cooling to 0° C. on ice.

The oligonucleotide sequences “A” and “AB” of the invention may be prepared using techniques that are standard or conventional in the art, or may be purchased from commercial sources.

Immobilization of the oligonucleotide primer population “A” to a support by the 5′ end leaves its 3′ end remote from the support such that the primer is available for chain extension by a polymerase once hybridization with a complementary sequence contained at the 3′ end of the nucleic acid template has taken place.

The distance between the individual oligonucleotide primers in a cluster and the individual nucleic acid templates (and hence the density of the primers and nucleic acid templates) can be controlled by altering the concentration of primers that are immobilized to the support. A preferred density of oligonucleotide primers is at least 1 fmol/mm², preferably at least 10 fmol/mm², more preferably between about 30 and about 60 fmol/mm². The density of nucleic acid templates for use in the method of the invention is typically about 10,000/mm² to about 100,000/mm². It is believed that higher densities, for example, about 100,000/mm² to about 1,000,000/mm² and about 1,000,000/mm² to about 10,000,000/mm² may be achieved.

Controlling the density of attached oligonucleotide primers and nucleic acid templates in turn allows the final density of nucleic acid clusters on the surface of the support to be controlled. This is due to the fact that according to the method of the invention, one nucleic acid coordinate can result from the attachment of one nucleic acid template, provided that the oligonucleotide primers of the invention are present in a suitable location on the solid support. The density of nucleic acid molecules within a single coordinate can also be controlled by controlling the density of attached oligonucleotide primers.

Once the oligonucleotide primers of the invention have been immobilized on the solid support at the appropriate density, nucleic acid clusters of the invention can then be generated by carrying out an appropriate number of cycles of amplification on the annealed bound template nucleic acid so that each cluster contains multiple copies of the original nucleic acid template and its complementary sequence. One cycle of amplification entails of the steps of hybridization, extension and denaturation. These steps are generally performed using reagents and conditions well known in the art for PCR.

A typical amplification reaction involves subjecting the solid support and attached nucleic acid template and “A” primers or colony primers to conditions which induce primer hybridization, for example subjecting them to a temperature of about 65° C. Under these conditions the sequence complementary to “A” or colony primer at the 3′ end of the nucleic acid template will hybridize to the immobilized oligonucleotide primer “A” or colony primer. In the presence of conditions and reagents to support primer extension, for example a temperature of about 72° C., the presence of a nucleic acid polymerase (for example, a DNA dependent DNA polymerase or a reverse transcriptase molecule (i.e., an RNA dependent DNA polymerase), or an RNA polymerase), plus a supply of nucleoside triphosphate molecules or any other nucleotide precursors, for example modified nucleoside triphosphate molecules, the oligonucleotide primer will be extended by the addition of nucleotides complementary to the annealed or covalently attached template nucleic acid sequence.

Examples of nucleic acid polymerases which can be used in the present invention include DNA polymerase (Klenow fragment, T4 DNA polymerase), heat-stable DNA polymerases from a variety of thermostable bacteria (such as Taq, VENT, Pfu, Tfl DNA polymerases) as well as their genetically modified derivatives (TaqGold, VENTexo, Pfu exo). A combination of RNA polymerase and reverse transcriptase can also be used to generate the amplification of a DNA colony. Preferably the nucleic acid polymerase used for colony primer extension is stable under PCR reaction conditions, i.e., repeated cycles of heating and cooling, and is stable at the denaturation temperature used, usually about 94° C. Preferably the DNA polymerase used is Taq DNA polymerase.

Preferably the nucleoside triphosphate molecules used are deoxyribonucleotide triphosphates, for example dATP, dTTP, dCTP, dGTP, or are ribonucleoside triphosphates for example dATP, dUTP, dCTP, dGTP. The nucleoside triphosphate molecules may be naturally or non-naturally occurring.

After the hybridization and extension steps, and upon subjecting the support and attached nucleic acids to denaturation conditions and washing, one nucleic acid sequence will be present, extended from the immobilized oligonucleotide primer “A” or colony primer. In the case of the “A” primer, the extended primer forming the “C” strand is then able to initiate further rounds of amplification on subjecting the support to one cycle of hybridization, extension and denaturation using oligonucleotide “AB”. “AB” is then washed away. Further cycles of hybridization, extension and denaturation require no additional oligonucleotide primers and will result in a nucleic acid coordinate containing multiple immobilized copies of the template nucleic acid and its complementary sequence. In the case of a colony primer, further rounds of amplification are initiated by annealing the attached oligonucleotide sequence Z to the colony primers as previously described.

The initial immobilization of the template nucleic acid means that the template nucleic acid can only hybridize with complementary primer located at a distance within the total length of the template nucleic acid. Thus the boundary of the nucleic acid colony formed is limited to a relatively local area in which the initial template nucleic acid was immobilized. The boundary of a nucleic acid coordinate is limited by the surface area containing covalently bound “A” primers. Clearly, once more copies of the template molecule and its complement have been synthesized by carrying out further rounds of amplification, i.e., further rounds of hybridization, extension and denaturation, the boundary of the nucleic acid colony being generated will be further extended. Regardless, the boundary of the coordinate is still limited to an area to which the initial nucleic acid template was immobilized.

The method of the present invention allows the generation of a nucleic acid cluster from a single annealed nucleic acid template and that the size of these clusters can be controlled by altering the number of rounds of amplification that the nucleic acid template is subjected to or by confining the surface area over which the “A” primers are attached. Thus, the number of nucleic acid coordinates formed on the surface of the solid support is dependent upon the number of oligonucleotide primers which are initially immobilized to the support. It is for this reason that preferably the solid support to which the oligonucleotide primers have been immobilized contains a micro-lawn of immobilized oligonucleotide primers at an appropriate density and at discrete, identifiable locations or coordinates on the solid support.

The methods for creating coordinates result, for example, in an array of specific oligonucleotide primers in particular local areas of the solid support. Initiating amplification by this method is not limited by the necessity of spotting specific nucleic acid templates at each of the local areas. The nucleic acids fragments used as the initial templates will locate their appropriate coordinates within the array by specifically annealing to only one of the “A” sequences. In this fashion, the approximately 100,000 template fragments will be arrayed on the solid support in precisely the same fashion as the oligonucleotide primers used to create each coordinate. Likewise, the amplification initiator (“AB”) will locate their respective template fragments also by specific annealing.

The method of creating colonies result, for example, in a lawn of colony primers covering the entire solid support. Nucleic acid amplification therefore results in a random array of colonies.

Once nucleic acid clusters have been generated, at least one an additional step, such as for example visualization or sequencing, can be carried out. DNA visualization might for example be required if it is necessary to screen the clusters generated for the presence or absence of for example the whole or part of a particular nucleic acid fragment. In this case the clusters which contain the particular nucleic acid fragment may be detected by designing a nucleic acid probe which specifically hybridizes to the nucleic acid fragment of interest.

Such a nucleic acid probe is preferably labeled with a detectable entity such as a fluorescent group, a biotin containing entity (which can be detected by for example an incubation with streptavidin labeled with a fluorescent group), a radiolabel (which can be incorporated into a nucleic acid probe by methods well known and documented in the art and detected by detecting radioactivity for example by incubation with scintillation fluid), or a dye or other staining agent.

Alternatively, such a nucleic acid probe may be unlabelled and designed to act as a primer for the incorporation of a number of labeled nucleotides with a nucleic acid polymerase. Detection of the incorporated label and thus the nucleic acid coordinates can then be carried out.

The nucleic acid clusters of the invention are then prepared for hybridization. Such preparation involves the treatment of the clusters so that all or part of the nucleic acid templates making up the clusters is present in a single stranded form. This can be achieved for example by heat denaturation of any double stranded DNA in the clusters. After preparation of the clusters for hybridization, the labeled or unlabeled probe is then added to the clusters under conditions appropriate for the hybridization of the probe with its specific DNA sequence. Such conditions may be determined by a person skilled in the art using known methods and will depend on for example the sequence of the probe.

The probe may then be removed by heat denaturation and, if desired, a probe specific for a second nucleic acid may be hybridized and detected. These steps may be repeated as many times as necessary or desired.

Labeled probes which are hybridized to nucleic acid colonies can then be detected using apparatus including an appropriate detection device. A preferred detection system for fluorescent labels is a charge-coupled device (CCD) camera, which can optionally be coupled to a magnifying device, for example a microscope. Using such technology many colonies may be simultaneously monitored in parallel. For example, using a microscope with a CCD camera and a 10× or 20× objective, colonies over a surface of between 1 mm² and 4 mm² may be observed, which corresponds to monitoring between 10,000 and 200,000 clusters in parallel.

An alternative method of monitoring the clusters generated entails scanning the surface covered with clusters. For example, systems in which up to 100,000,000 clusters could be arrayed simultaneously and monitored by taking pictures with the CCD camera over the whole surface can be used. In this fashion, as many as about 100,000,000 clusters can be monitored in a short time.

Any other devices allowing detection and preferably quantification of fluorescence on a surface may be used to monitor the nucleic acid clusters of the invention. For example fluorescent imagers or confocal microscopes could be used.

If the labels are radioactive then a radioactivity detection system is required.

In practicing the present invention, amplified reference or target DNA, bound to the solid-phase matrix, is hybridized with a second DNA sample under conditions that favor the formation of mismatch loops. Both DNA samples are purified and processed in the same fashion by, for example, digesting with the same restriction enzyme(s). In a preferred embodiment, one of the population of DNA fragments, either reference or target, is amplified by, for example, PCR, in solution. In order to saturate the annealing sites on the solid support, the amount of unbound DNA fragments generated using PCR in solution is preferably in a molar excess over the reference fragments attached to the solid support. Amplification of target DNA fragments may be primed by, for example, “A” and “AB” primers, “A” and “B” primers or “Z” and “Y” primers. In a preferred embodiment, at least one of the PCR primers used to amplify the unbound DNA sample is detectibly labeled with, for example, a fluorescent moiety. The detectable label is preferably a fluorophore and the linker is preferably an acid labile linker, a photolabile linker, or can contain a disulphide linkage. The purpose of having a detectable label on one or both primers is to provide for the detection of annealing between unbound DNA and DNA attached to the solid support. In instances where annealing fails to occur at a particular cluster, there will be no fluorescent signal. In that situation, the DNA fragment(s) that are complementary to the DNA at that coordinate position can be sequenced in their entirety by traditional sequencing techniques rather than by mismatch-initiated sequencing. Failed amplifications of DNA restriction fragments, for example, can occur for example in certain types of restriction fragment length polymorphisms (RFLPs). In the case of an additional restriction site within an unbound DNA fragment, amplification of that fragment will not occur. The two restriction fragments complementary to the bound DNA cluster will have to be obtained by other means such as molecular cloning in suitable vectors after PCR amplification of the entire fragment from genomic DNA that has not been digested with a restriction enzyme.

In another embodiment, multiple arrays are prepared comprising coordinates each containing restricted DNA fragments derived from different restriction enzymes. A comparison of different arrays derived from different restriction enzymes will enable the detection of RFLPs (i.e., SNPs that occur at restriction sites).

Genomes may include multiple prevalent versions, which contain alterations in sequence relative to each other that cause no discernable pathological effect. Such variations are designated “polymorphisms” or “allelic variants”. Most preferably, genomic DNA from a single individual is used for the second DNA sample of unknown sequence. This insures that, statistically, hybrids formed between the first and second DNA sample will be perfectly matched except in the region of the mutation, where discrete mismatch regions will form. In some applications, it is desired to detect polymorphisms. In these cases, appropriate sources for the second DNA sample will be selected accordingly. Depending upon what method is used subsequently to detect mismatches, the unknown DNA may also be chemically or enzymatically modified, e.g., to remove or add methyl groups. Likewise, the immobilized reference DNA can also be chemically modified.

Hybridization reactions according to the present invention may be performed in solutions ranging from about 10 mM NaCl to about 600 mM NaCl, and at temperatures ranging from about 37° C. to about 65° C. It will be understood that the stringency of a hybridization reaction is determined by both the salt concentration and the temperature. Thus, a hybridization performed in 10 mM salt at 37° C. may be of similar stringency to one performed in 500 mM salt at 65° C. For the purposes of the present invention, any hybridization conditions may be used that form perfect hybrids between precisely complementary sequences and mismatch loops between non-complementary sequences in the same molecules. Preferably, hybridizations are performed in about 600 mM NaCl at about 65° C. Following the hybridization step, DNA molecules that have not hybridized to the target DNA sample are removed by washing under stringent conditions, e.g., 0.1×SSC at 65° C.

The hybrids formed by the hybridization reaction may then be treated to block any free ends so that they cannot serve as substrates for further enzymatic modification such as, e.g., by RNA ligase. Suitable blocking methods include without limitation removal of 5′ phosphate groups, homopolymeric tailing of 3′ ends with dideoxynucleotides, and ligation of modified double-stranded oligonucleotides to the ends of the duplex.

### MISMATCH RECOGNITION AND CLEAVAGE

The hybrids are treated so that one or both DNA strands are cleaved within, or in the vicinity of, the mismatch region. Depending on the method used for mismatch recognition and cleavage (see below), cleavage may occur at some predetermined distance from either boundary of the mismatch region, and may occur on the unknown or reference strand. The “vicinity” of the mismatch as used herein thus encompasses from 1 to about 2000 bases from the borders of the mismatch. Non-limiting examples of mismatch recognition and cleavage systems suitable for use in the present invention include nicking proteins, mismatch repair proteins, nucleotide excision repair proteins, chemical modification, and combinations thereof. These embodiments are described below.

In general, the mismatch recognition and/or modification proteins necessary for each embodiment described below are isolated using methods that are well known to those skilled in the art. Preferably, when the sequence of a genome is known, the restriction sites are also known so that the restriction fragments can be amplified using adjacent sequences as primer sites.

The mismatch recognition and modification proteins used in practicing the present invention may be derived from any species, from E. coli to humans, or mixtures thereof. Typically, functional homologs for a given protein exist across phylogeny. A “functional homolog” of a given protein as used herein is another protein that can functionally substitute for the first protein, either in vivo or in a cell-free reaction.

Mismatch repair proteins:

A number of different enzyme systems exist across phylogeny to repair mismatches that form during DNA replication. In E. coli, one system involves the MutY gene product, which recognizes A/G mismatches and cleaves the A-containing strand (Tsai-Wu et al., J. Bacteriol. 178:1902, 1991). Another system in E. coli utilizes the coordinated action of the MutS, MutL, and Mutes proteins to recognize errors in newly-synthesized DNA strands specifically by virtue of their transient state of under-methylation (prior to their being acted upon by dam methylase in the normal course of replication). Cleavage typically occurs at a hemi-methylated GATC site within 1-2 kb of the mismatch, followed by exonucleolytic cleavage of the strand in either a 3′-5′ or 5′-3′ direction from the nick to the mismatch. In vivo, this is followed by re-synthesis involving DNA polymerase III holoenzyme and other factors (Cleaver, Cell, 76:1-4, 1994).

Mismatch repair proteins for use in the present invention may be derived from E. coli (as described above) or from any organism containing mismatch repair proteins with appropriate functional properties. Non-limiting examples of useful proteins include those derived from Salmonella typhimurium (MutS, MutL); Streptococcus pneumoniae (HexA, HexB); Saccharomyces cerevisiae (“all-type”, MSH2, MLH1, MSH3); Schizosaccharomyces pombe (SWI4); mouse (rep1, rep3); and human (“all-type”, hMSH2, hMLH1, hPMS1, hPMS2, duel). Preferably, the “all-type” mismatch repair system from human or yeast cells is used (Chang et al., Nuc. Acids Res. 19:4761, 1991; Yang et al., J. Biol. Chem. 266:6480, 1991). In a preferred embodiment, heteroduplexes formed between reference DNA and unknown DNA as described above are incubated with human “all-type” mismatch repair activity that is purified essentially as described in International Patent Application WO/93/20233. Incubations are performed in, e.g., 10 mM Tris-HCl pH 7.6, 10 mM ZnCl₂, 1 mM dithiothreitol, 1 mM EDTA and 2.9% glycerol at 37° C. for 1-3 hours. In another embodiment, purified MutS, MutL, and MutH are used to cleave mismatch regions (Su et al., Proc. Natl. Acad. Sci. USA 83:5057, 1986; Grulley et al., J. Biol. Chem. 264:1000, 1989).

In a preferred embodiment mismatches result in nicking activity on one of the strands in the immediate vicinity of the mismatch, preferably between the mismatched nucleotide and the next nucleotide on the 5′ side. The all-type nicking enzyme (ATE) from human HeLa cells or calf thymus can nick DNA at the first phosphodiester bond 5′ to all 8 possible mismatched bases. The strand disparity of this nicking is influenced by the neighboring nucleotide sequences. After nicking, the ATE covalently binds the 3′ end of the DNA product to form a cleavable complex. Topoisomerases I introduce transient DNA single-strand breaks by forming a catalytic intermediate in which a covalent bond is generated between an enzyme tyrosine residue (Tyr723 for human topoisomerase I) and the 3′-end of the broken DNA. In a further preferred embodiment tyrosyl-DNA phosphodiesterase-1 (Tdp1) then removes tyrosine from complexes in which the amino acid is linked to the 3′-end of DNA fragments. Polynucleotide kinase phosphatase is then used to regenerate the 3′ hydroxyl to create a substrate for DNA polymerase immediately 5′ of the mismatch.

In a further preferred embodiment, the entire population of clusters containing annealed double stranded DNA is first treated with an appropriate topoisomerase I or combination of topoisomerases in order to nick one of the DNA strands in the population of nucleic acids comprising a cluster. The topoisomerase used for this nicking is itself derivatized with a fluorescent compound. Methods for derivatizing proteins with detectible compounds while leaving the enzymatic activity of the protein intact are well known to those of skill in the art. The resulting covalent attachment of fluorescent topoisomerases identifies those coordinates or colonies that contain mismatched nucleotides and indicates which restriction fragments could be candidates for further sequence analysis. The remaining coordinates or colonies do not contain mismatches and therefore the target DNA and the reference DNA in those restriction fragments are exactly the same and are contained accurately in the reference DNA sequence database. In other words, the sequence of the unknown DNA in a cluster which was not labeled by, for example, topoisomerase I, is now known.

Fragments that contain identifiable mismatches can be sequenced in their entirety to identify the specific mismatch nucleotide and its location. The sequence of the particular identified restriction fragments can be obtained after, for example, PCR amplification of target DNA using primers derived from the sequence database. The sequencing can be carried out using standard methods such as, for example, the dideoxy terminator nucleotide method and analysis on an ABI 377 sequencer. Alternatively, the amplified restriction fragment can be evaluated for binding of any of the known SNPs using standard oligonucleotide hybridization techniques. In cases where oligonucleotide binding identifies a particular SNP, further restriction digestion of the restriction fragment using additional restriction enzymes can be carried out to further narrow down the location of the SNP.

Nucleotide excision repair proteins:

In E. coli, four proteins, designated UvrA, UvrB, UvrC, and UvrD, interact to repair nucleotides that are damaged by UV light or otherwise chemically modified (Sancar, Science 266: 1954, 1994), and also to repair mismatches (Huang et al., Proc. Natl. Acad. Sci. USA 91:12213, 1994). UvrA, an ATPase, makes an A₂ B₁ complex with UvrB, binds the site of the lesion, unwinds and kinks the DNA, and causes a conformational change in UvrB that allows it to bind tightly to the lesion site. UvrA then dissociates from the complex, allowing UvrC to bind. UvrB catalyzes an endonucleolytic cleavage at the fifth phosphodiester bond 3′ from the lesion; UvrC then catalyzes a similar cleavage at the eighth phosphodiester bond 5′ from the lesion. Finally, UvrD (helicase II) releases the excised oligomer. In vivo, DNA polymerase I displaces UvrB and fills in the excision gap, and the patch is ligated.

In one embodiment of the present invention, heteroduplexes formed between unknown DNA and reference DNA are treated with a mixture of UvrA, UvrB, UvrC, with or without UvrD. As described above, the proteins may be purified from wild-type E. coli, or from E. coli or other appropriate host cells containing recombinant genes encoding the proteins, and are formulated in compatible buffers and concentrations. The final product is a heteroduplex containing a single-stranded gap covering the site of the mismatch.

Excision repair proteins for use in the present invention may be derived from E. coli (as described above) or from any organism containing appropriate functional homologs. Non-limiting examples of useful homologs include those derived from S. cerevisiae (RAD1, 2, 3, 4, 10, 14, and 25) and humans (XPF, XPG, XPD, XPC, XPA, ERCC1, and XPB) (Sancar, Science 266:1954, 1994). When the human homologs are used, the excised patch comprises an oligonucleotide extending 5 nucleotides from the 3′ end of the lesion and 24 nucleotides from the 5′ end of the lesion. Aboussekhra et al. (Cell 80:859, 1995) disclose a reconstituted in vitro system for nucleotide excision repair using purified components derived from human cells.

Chemical Mismatch Recognition:

Heteroduplexes formed between unknown DNA and reference DNA according to the present invention may be chemically modified by treatment with osmium tetroxide (for mispaired thymidines) and hydroxylamine (for mispaired cytosines), using procedures that are well known in the art (see, e.g., Grompe, Nature Genetics 5:111, 1993; and Saleeba et al., Meth. Enzymol. 217:288, 1993). In one embodiment, the chemically modified DNA is contacted with excision repair proteins (as described above). The hydroxylamine- or osmium-modified bases are recognized as damaged bases in need of repair, one of the DNA strands is selectively cleaved, and the product is a gapped heteroduplex as above.

Resolvases:

Resolvases are enzymes that catalyze the resolution of branched DNA intermediates that form during recombination events (including Holliday structures, cruciforms, and loops) via recognition of bends, kinks, or DNA deviations (Youil et al., Proc. Natl. Acad. Sci. USA 92:87, 1995). For example, Endonuclease VII derived from bacteriophage T4 (T4E7) recognizes mismatch regions of from one to about 50 bases and produces double-stranded breaks within six nucleotides from the 3′ border of the mismatch region. T4E7 may be isolated from, e.g., a recombinant E. coli that over-expresses gene 49 of T4 phage (Kosak et al., Eur. J. Biochem. 194:779, 1990). Another suitable resolvase for use in the present invention is Endonuclease I of bacteriophage T7 (T7E1), which can be isolated using a polyhistidine purification tag sequence (Mashal et al., Nature Genetics 9:177, 1995).

In a preferred embodiment, heteroduplexes formed between patients' DNA and wild-type DNA as described above are incubated in a 50 μl reaction with 100-3000 units of T4E7 for 1 hour at 37° C.

### SEQUENCE DETERMINATION

In one embodiment of the present invention, immobilized target DNA from an individual is annealed to reference DNA to form mismatch regions and then treated with mismatch nicking proteins, mismatch repair proteins, excision repair proteins, chemical modification and cleavage reagents, or combinations of such agents. This treatment introduces single-stranded breaks at predetermined locations on one or both sides of a mismatch region and may cause the selective excision of single-stranded fragment covering the mismatch region. Alternatively, the treatment results in a single nick being introduced at the 5′ end of the mismatch. The resulting structure is a nicked or gapped heteroduplex in which the gap may be from about 5 to about 2000 bases in length, depending on the mismatch recognition system used. In the case of a nick, no gap is formed but a free 3′ hydroxyl is present at the site of the mismatch.

In methods of the present invention wherein the additional step of performing at least one step of sequence determination of at least one of the nucleic acid clusters generated is performed, the sequence determination may be carried out using any appropriate solid phase sequencing technique. For example, one technique of sequence determination that may be used in the present invention involves hybridizing an appropriate primer, sometimes referred to herein as a “sequencing primer”, with the nucleic acid template to be sequenced, extending the primer and detecting the nucleotides used to extend the primer. Preferably the nucleic acid used to extend the primer is detected before a further nucleotide is added to the growing nucleic acid chain, thus allowing base-by-base in situ nucleic acid sequencing.

Specially designed nucleotides with fluorescent reversible 3′ terminators allow each cycle of a sequencing reaction to occur simultaneously for all coordinates in the presence of all four nucleotides (A, C, T, and G). In each cycle, the polymerase is able to select the correct base to incorporate, with the natural competition among all four alternatives leading to higher accuracy than methods where only one nucleotide is present in the reaction mix at a time. Sequences where a particular base is repeated (e.g., homopolymers) are addressed like any other sequence and resolved with high accuracy. The simultaneous sequencing of the thousands of clusters present on the solid support is accomplished by recording the unique fluorescent signal for each nucleotide at each position during every cycle of the process. After recording, the fluorescent terminators are removed, e.g., by a chemical reaction for example by the addition of a low pH solution such that the next round of polymerase additions can proceed.

In cases where there are multiple mismatches between target and reference DNA present at a specific cluster, the sequencing signal may be uninterpretable. In those situations, the target DNA at that cluster can be sequenced in its entirety using traditional sequencing techniques rather than mismatch-directed sequencing.

The detection of incorporated nucleotides is facilitated by including one or more labeled nucleotides in the primer extension reaction. Any appropriate detectable label may be used, for example a fluorophore, radiolabel etc. Preferably a fluorescent label is used. The same or different labels may be used for each different type of nucleotide. Where the label is a fluorophore and the same labels are used for each different type of nucleotide, each nucleotide incorporation can provide a cumulative increase in signal detected at a particular wavelength. If different labels are used then these signals may be detected at different appropriate wavelengths. If desired, a mixture of labeled and unlabelled nucleotides is provided.

In order to allow the hybridization of an appropriate sequencing primer to the nucleic acid template to be sequenced, the nucleic acid template should normally be in a single stranded form. If the nucleic acid templates making up the nucleic acid colonies are present in a double stranded form these can be processed to provide single stranded nucleic acid templates using methods well known in the art, for example by denturation, cleavage etc.

The sequencing primers which are hybridized to the nucleic acid template and used for primer extension are preferably short oligonucleotides, for example of 15 to 25 nucleotides in length. The sequence of the primers is designed so that they hybridize to part of the nucleic acid template to be sequenced, preferably under stringent conditions. The sequence of the primers used for sequencing may have the same or similar sequences to that of the colony primers used to generate the nucleic acid colonies of the invention. The sequencing primers may be provided in solution or in an immobilized form.

Once the sequencing primer has been annealed to the nucleic acid template to be sequenced by subjecting the nucleic acid template and sequencing primer to appropriate conditions, determined by methods well known in the art, primer extension is carried out, for example using a nucleic acid polymerase and a supply of nucleotides, at least some of which are provided in labeled form, and conditions suitable for primer extension if a suitable nucleotide is provided. Examples of nucleic acid polymerases and nucleotides which may be used are described above.

Preferably after each primer extension step a washing step is included in order to remove unincorporated nucleotides which may interfere with subsequent steps. Once the primer extension step has been carried out, the nucleic acid colony is monitored in order to determine whether a labeled nucleotide has been incorporated into an extended primer. The primer extension step may then be repeated in order to determine the next and subsequent nucleotides incorporated into an extended primer.

In one embodiment of the present invention, no sequencing primer is used to initiate sequencing reaction. In this instance, the gap or nick created by the nicking or mismatch repair proteins is used as the primer to initiate addition of nucleotides to an exposed 3′ hydroxyl group near the site of the mismatch. The polymerase catalyzed extension from the 3′ hydroxyl continues through the mismatch site in order to obtain the sequence of DNA in the vicinity of and including the mismatch site. In a preferred embodiment, the exposed 3′ hydroxyl is immediately adjacent to the mismatch nucleotide on the 5′ side of the mismatch. This nicking activity can be achieved by, for example, an ATE enzyme capable of nicking at all eight mismatch pairs. An example of an ATE enzyme is a topoisomerase I. Topoisomerase I enzymes can be obtained from a wide variety of eukaryotic and bacterial sources. In general, a particular topoisomerase I enzyme will exhibit a strand preference in its nicking activity and will always nick a particular strand at the site of the mismatch. The complementary strand is then not a substrate for topoisomerase I nicking activity. Some topoisomerase I enzymes pick a strand for nicking based on preference for a local sequence compared to the complementary sequence. Some topoisomerase I enzymes simply pick one particular strand in the DNA major groove.

Within a population of double stranded nucleic acids as, for example, in a nucleic acid cluster of the present invention, a particular topoisomerase I will only nick one strand. The sequence of the DNA in the vicinity of and including the mismatch cannot unambiguously determine the exact composition of the mismatch if, for instance, the reference strand is different from the database at the mismatch site. In addition, if the target DNA is sequenced at the mismatch, that sequence will simply be the result of the correction of the mismatch by the addition of a nucleotide complementary to the reference strand. In order to overcome this ambiguity, it is advantageous to sequence both strands of the DNA at the mismatch site. To accomplish this, both strands of the DNA contained in a coordinate need to be nicked or have gaps. This would result in, for example, 50% of the reference strand and 50% of the complementary target strand being nicked or gapped at the mismatch site or in the vicinity of the mismatch site. The appropriate combination of topoisomerase I enzymes from different species or of topoisomerase I enzymes combined with other mismatch repair or nucleotide excision repair proteins will accomplish this. The appropriate combination of proteins to accomplish this can be determined by sequencing the DNA from each respective nick or gap.

The resulting fluorescent signals from the sequencing initiated on different strands or even at different positions on different strands are therefore derived from two different nucleotides and two different fluorophors at each step of the sequencing progression or on each discrete fragment of terminated fluorescent DNA. In a preferred embodiment, the fluorescent groups on the nucleotide comprise removable terminators. Since the reference strand sequence on the coordinate restriction fragment is known, the sequence of the reference strand and its complementary target strand can be determined from the binary sequence derived from the detection of two different fluors at each sequencing position at the same time. As an illustration, if dATP, dTTP, dGTP, and dCTP nucleotides were modified at either the ribose 3′ position or a position on the nucleotide base by fluorescent groups that have emission wavelengths of 400, 500, 600, and 700 nm respectively, ten possible nucleotide pairings in DNA (eight mismatches and two complementary pairings) arise, namely A/T, A/G, A/C, T/G, T/C, G/C, A/A, T/T, G/G, and C/C. The binary fluorescent signals from each pairing derived from the combinations of the individual fluors would be (in nm) 400+500, 400+600, 400+700, 500+600, 500+700, 600+700, 400 only, 500 only, 600 only, and 700 only, respectively. Each wavelength or pairing is readily distinguishable from the others with the use of appropriate excitation wavelengths as well as appropriate emission detection filters.

In another embodiment, the potential ambiguity of sequencing a target or reference strand at the site of a possible mismatch is clarified by annealing the appropriate oligonucleotides to the putative mismatch or SNP site. For example, target DNA or fragments of target DNA, or PCR products from target DNA are annealed with three oligonucleotides either in three separate reactions or using oligonucleotides that are distinguishable (e.g., by virtue of containing distinguishable fluorescent groups) in one reaction. The annealing conditions are chosen such that the oligonucleotide which is perfectly complementary to the mismatch or SNP on the target DNA is the only oligonucleotide which binds to the DNA. Annealing conditions that allow complementary oligonucleotide binding but do not allow binding of oligonucleotides with a single mismatch generally depend primarily on the appropriate temperature for annealing. For example, the 15 base oligonucleotide 5′ACGACAGGTTTACCA3′ has a range of Tm (melting temperatures) from 48° C. to 62° C. depending on the Na+ ion concentration in the annealing solution. A mismatch at nucleotide 9 however could lower the Tm by 3° C. compared to the perfectly complementary oligonucleotide under the same conditions. Therefore, by adjusting temperature and the Na+ concentration, conditions can be found that allow binding of a perfectly complementary oligonucleotide and prevent binding of a mismatched oligonucleotide to the target DNA.

Any device allowing detection and preferably quantification of the appropriate label, for example fluorescence or radioactivity, may be used for sequence determination. If the label is fluorescent a CCD camera optionally attached to a magnifying device (as described above), may be used. In fact the devices used for the sequence determining aspects of the present invention may be the same as those described above for monitoring the amplified nucleic acid colonies.

The detection system is preferably used in combination with an analysis system in order to determine the number and nature of the nucleotides incorporated at each cluster after each step of primer extension. This analysis, which may be carried out immediately after each primer extension step, or later using recorded data, allows the sequence of the nucleic acid template within a given cluster to be determined.

If the sequence being determined is unknown, the nucleotides applied to a given cluster are usually applied in a chosen order which is then repeated throughout the analysis, for example dATP, dTTP, dCTP, dGTP. If, however, the sequence being determined is known and is being re-sequenced, for example to analyze whether or not small differences in sequence from the known sequence are present, the sequencing determination process may be made quicker by adding the nucleotides at each step in the appropriate order, chosen according to the known sequence. Differences from the given sequence are thus detected by the lack of incorporation of certain nucleotides at particular stages of primer extension. Thus full or partial sequences of the amplified nucleic acid templates making up particular nucleic acid colonies may be determined using the methods of the present invention.

In a further embodiment of the present invention, the full or partial sequence of more than one nucleic acid can be determined by determining the full or partial sequence of the amplified nucleic acid templates present in more than one nucleic acid coordinate. Preferably a plurality of sequences is determined simultaneously.

Reliability of the sequence determination of nucleic acids using the methods of the present invention is enhanced due to the fact that large numbers of each nucleic acid to be sequenced are provided within each nucleic acid coordinate of the invention. If desired, further improvements in reliability can be obtained by providing a plurality of nucleic acid colonies containing the same nucleic acid template to be sequenced, then determining the sequence for each of the plurality of colonies and comparing the sequences thus determined.

Preferably the attachment of the oligonucleotide primer as well as the extended nucleic acid template on the solid support is thermostable at the temperature to which the support may be subjected to during the nucleic acid amplification reaction, for example temperatures of up to approximately 100° C., for example approximately 94° C. Preferably the attachment is covalent in nature.

To determine the nucleotide sequence of the nicked or excised region (including the mismatch), the heteroduplexes are incubated with an appropriate DNA polymerase enzyme in the presence of dideoxynucleotides. Suitable enzymes for use in this step include without limitation DNA polymerase I, DNA polymerase III holoenzyme, T4 DNA polymerase, and T7 DNA polymerase. The only requirement is that the enzyme be capable of accurate DNA synthesis using the gapped heteroduplex as a substrate. The presence of dideoxynucleotides, as in a Sanger sequencing reaction, insures that a nested set of premature termination products will be produced, and that resolution of these products by, e.g., gel electrophoresis, will display the DNA sequence across the gap.

High-Throughput Applications

The methods of the present invention are particularly suitable for high-throughput analysis of DNA, i.e., the rapid and simultaneous processing of genomic DNAs derived from an individual. Furthermore, in contrast to other methods for de novo mutation detection, the methods of the present invention are suitable for the simultaneous analysis of a large number of restriction fragments in a single reaction. This is referred to as “multiplex” analysis. The manipulations involved in practicing the methods of the present invention lend themselves to automation, e.g., using multiwell formats as a solid support or as a receptacle for, e.g., beads; robotics to perform sequential incubations and washes; and, finally, automated sequencing using commercially available automated DNA sequencers.

For use of the present invention in diagnostics and screening, whole genomes or fractions of genomes may be amplified into colonies for DNA sequencing of known single nucleotide polymorphisms (SNP). SNP identification has application in medical genetic research to identify genetic risk factors associated with diseases. SNP genotyping will also have diagnostic applications in pharmaco-genomics for the identification and treatment of patients with specific medications.

For use of the present invention in genetic diversity profiling, populations of for example organisms or cells or tissues can be identified by the amplification of the sample DNA into coordinates, followed by the DNA sequencing of the specific “tags” for each individual genetic entity. In this way, the genetic diversity of the sample can be defined by counting the number of tags from each individual entity.

For use of the present invention in gene expression monitoring, the expressed mRNA molecules of a tissue or organism under investigation are converted into cDNA molecules which are amplified into sets of colonies for DNA sequencing. The frequency of coordinates coding for a given mRNA is proportional to the frequency of the mRNA molecules present in the starting tissue. Applications of gene expression monitoring are in biomedical research.

A whole genome slide, where the entire genome of a living organism is represented in a number of DNA colonies numerous enough to contain all the sequences of that genome may be prepared using the methods of the invention. The genome slide is the genetic card of any living organism. Genetic cards have applications in medical research and genetic identification of living organisms of industrial value.

The present invention may also be used to carry out whole genome sequencing where the entire genome of a living organism is amplified as sets of coordinates for extensive DNA sequencing. Whole genome sequencing allows for example, 1) a precise identification of the genetic strain of any living organism; 2) discovery of novel genes encoded within the genome and 3) discovery of novel genetic polymorphisms.

The applications of the present invention are not limited to an analysis of nucleic acid samples from a single organism/patient. For example, nucleic acid tags can be incorporated into the nucleic acid templates and amplified, and different nucleic acid tags can be used for each organism/patient. Thus, when the sequence of the amplified nucleic acid is determined, the sequence of the tag may also be determined and the origin of the sample identified.

Thus, a further aspect of the invention provides the use of the methods of the invention, or the nucleic acid colonies of the invention, or the plurality of nucleic acid templates of the invention, or the solid supports of the invention, for providing nucleic acid molecules for sequencing and re-sequencing, gene expression monitoring, genetic diversity profiling, diagnosis, screening, whole genome sequencing, whole genome polymorphism discovery and scoring and the preparation of whole genome slides (i.e., the whole genome of an individual on one support), or any other applications involving the amplification of nucleic acids or the sequencing thereof.

A yet further aspect of the invention provides a kit for use in sequencing, re-sequencing, gene expression monitoring, genetic diversity profiling, diagnosis, screening, whole genome sequencing, whole genome polymorphism discovery and scoring, or any other applications involving the amplification of nucleic acids or the sequencing thereof. This kit contains a plurality of nucleic acid templates and colony primers of the invention bound to a solid support, as outlined above.

Citations of Publications Referenced Herein

Kruglyak, L. 1999. Nat. Genet. 22:139-144.

Risch, N., and Merikangas, K. 1996. Science 273:1516-1517.

Lu and Hsu, Genomics 14:249-255 1992.

Su et al. Genome 31:104-111 1992.

Landegren U et al., Science, 241:1077-1080, 1988.

Mashal et al., Nature Genetics, 9:177, 1995.

Maxam A M et al., Methods Enzymol., 65:499-560, 1980.

Mayall et al., J. Med. Genet., 27:658, 1990.

Meyers R M et al., Nature, 313:495-498, 1985.

Newton C R et al., Nuc Acids Res., 17:2503-2516, 1989.

Orita M et al., Proc. Natl. Acad. Sci. USA, 86:2766-2770, 1989.

Pease et al., Proc. Natl. Acad. Sci. USA, 91:5022, 1994.

Richards et al., Human Mol. Gen., 2:159, 1993.

Rommens et al., Am. J. Genet., 46:395-396, 1990.

Saleeba et al., Meth. Enzymol., 217:288, 1993.

Sancar, Science, 266:1954, 1994.

Shuber et al., Human Molecular Genetics, 2:153-158, 1993.

Sokolov, B P, Nucl. Acids Res., 18:3671, 1989.

Southern, E. M., J. Mol. Biol., 98:503-517, 1975.

Su et al., Proc. Natl. Acad. Sci. USA, 83:5057, 1986.

Thompson and Thompson, Genetics in Medicine, 5th Ed.

Tsai-Wu et al., J. Bacteriol., 178:1902, 1991.

Wallace R B et al., Nucl. Acids Res., 9:879-895, 1981.

Yeh et al., J. Biol. Chem., 266:6480, 1991.

Youil et al., Proc. Natl. Acad. Sci. USA, 92:87, 1995.

Aboussekhra et al., Cell 80:859, 1995.

Chang et al., Nuc. Acids Res. 19:4761, 1991.

Chehab et al., Nature, 329:293-294, 1987.

Cleaver, Cell, 76:1-4, 1994.

Cohen L B et al., Nature, 334:119-121, 1988.

Cotton R G E et al., Proc. Natl. Acad. Sci., 85:4397-4401, 1988.

Grilley et al., J. Biol. Chem., 264:1000, 1989.

Ealiassos et al., Nucleic Acids Research, 17:3606, 1989.

Huang et al., Proc. Natl. Acad. Sci. USA, 91:12213, 1994.

Keen J. et al., Trends Genet., 7:5, 1991.

Kosak et al., Eur. J. Biochem., 194:779, 1990.

All publications cited in the specification, both patent publications and non-patent publications are indicative of the level of skill of those skilled in the art to which this invention pertains. Any publication not already incorporated by reference herein is herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of sequencing nucleic acid, comprising:
a) preparing single stranded fragments of a first nucleic acid having a substantially known sequence, wherein each of the fragments has a substantially known sequence;
b) preparing single stranded fragments of a second nucleic acid having an unknown sequence;
c) contacting the single stranded fragments of a) or amplification products (copies) thereof, and the single stranded fragments of b) or copies thereof under conditions that allow formation of heterohybrid nucleic acid, wherein the heterohybrid nucleic acid comprises perfectly complementary heterohybrid nucleic acid and heterohybrid nucleic acid containing a mismatch;
d) distinguishing formation of heterohybrid nucleic acid containing a mismatch from formation of heterohybrid nucleic acid which is perfectly complementary; and
e) determining sequences of the mismatches in d), thus allowing elucidation of the sequence of the second nucleic acid.

2. The method of claim 1, wherein the single stranded fragments of the first nucleic acid and the single stranded fragments of the second nucleic acid are prepared by reacting the first and second nucleic acids with first and second restrictive endonucleases, which may be the same or different.

3. The method of claim 2, wherein the first and second restrictive endonucleases are the same.

4. The method of claim 1 wherein the single stranded fragments of the first nucleic acid or copies thereof, or wherein the single stranded fragments of the second nucleic acid, or copies thereof, are attached to a solid support.

5. The method of claim 4, wherein the single stranded fragments of the first nucleic acid, or the single stranded fragments of the second nucleic acid are amplified prior to being attached to the solid support.

6. The method of claim 4, wherein the single stranded fragments of the first nucleic acid, or the single stranded fragments of the second nucleic acid are amplified after being attached to the solid support.

7. The method of claim 4, wherein each of the amplified fragments comprises 5′ and 3′ flanking sequences of known sequence that serve as primers.

8. The method of claim 4, wherein copies of the single stranded fragments of the first nucleic acid, or copies of the single stranded fragments of the second nucleic acid, are attached to the solid support in the form of colonies.

9. The method of claim 8, wherein the single stranded fragments of the first nucleic acid, or the single stranded fragments of the second nucleic acid are templates which comprise, at their 5′ end, means for attachment to the solid support, and at their 3′ end, a sequence that hybridizes to a 3′ end of a colony primer, wherein the 5′ end of the colony primer comprises means for attachment to the solid support, and wherein the attachment comprises reacting the templates and the colony primers in the presence of the support such that the 5′ ends of the templates and the colony primers become attached to the solid support, and performing at least one round of nucleic acid amplification reaction on the attached templates, thus creating individual colonies of each of the amplified templates.

10. The method of claim 9, further comprising sequencing at least a portion of the amplified templates in each colony to allow for identification of a particular single stranded fragment contained in each colony.

11. The method of claim 1, wherein the single stranded fragments of the second nucleic acid, or copies thereof, are affixed to a solid support, and wherein the single stranded fragments of the first nucleic acid which contain a single nucleotide polymorphism (SNP) are labeled, such that annealing of the labeled single stranded fragment of the first nucleic acid that contains the SNP to a single stranded fragment of the second nucleic acid indicates the presence of the SNP in the single stranded fragment of the second nucleic acid.

12. The method of claim 1, wherein the single stranded fragments of the first nucleic acid, or copies thereof, or the single stranded fragments of the second nucleic acid, or copies thereof, are attached to a solid support, at a known location thereof, which comprises a coordinate.

13. The method of claim 4, wherein the solid support comprises a glass surface and the single stranded fragments of the first nucleic acid or copies thereof, or the single stranded fragments of the second nucleic acid, or copies thereof, are covalently attached to the glass surface.

14. The method of claim 4, wherein the single stranded fragments of the first or second nucleic acid which are not attached to the solid support are amplified via PCR with at least one detectably labeled PCR primer.

15. The method of claim 1, wherein (d) comprises contacting the heterohydrid nucleic acid formed in (c) with a mismatch nicking protein.

16. The method of claim 15, wherein the mismatch nicking protein comprises an all-type nicking enzyme (ATE).

17. The method of claim 16, wherein the ATE comprises Topoisomerase I.

18. The method of claim 16, wherein the ATE is detectably labeled.

19. The method of claim 1, wherein (e) comprises contacting the heterohydrid nucleic acid of (d) with at least one of a mismatch repair protein, an excision repair protein, a chemical modification reagent, or a chemical cleavage reagent.

20. The method of claim 1, wherein (e) comprises sequences both strands of the heterohybrid nucleic acid at the site of a mismatch.

21. A solid support comprising a plurality of coordinates, wherein each coordinate comprises a cluster of amplified single stranded fragments of a nucleic acid attached to the support at the coordinate, wherein at least a portion of the sequence of the fragments is known.

22. The solid support of claim 21, wherein each of the fragments comprises 5′ and 3′ primers, the sequences of which are known.

23. The solid support of claim 21, wherein the entire sequence of each of the fragments is known.
